(19) United States
(12) Patent Application Publication
(10) Pub. No.: US 2024/0074656 A1
(43) Pub. Date: Mar. 7, 2024

(54) METHOD AND APPARATUS FOR ENHANCED FULL-FIELD STIMULUS THRESHOLD (FST) AND PUPILLOMETRY SENSITIVITY THRESHOLD (PST) TESTING

(71) Applicant: Diagnosys LLC, Lowell, MA (US)

(72) Inventors: Jeffrey D. Farmer, Chelmsford, MA (US); Anna-Rosa Gejlsbjerg, Lowell, MA (US); Richard Robson, Holt (GB)

(21) Appl. No.: 18/139,069

(22) Filed: Apr. 25, 2023

Related U.S. Application Data
(60) Provisional application No. 63/334,387, filed on Apr. 25, 2022.

(51) Int. Cl. A61B 3/11; A61B 3/00; A61B 3/06

(52) U.S. Cl. CPC A61B 3/112; A61B 3/0008; A61B 3/0025; A61B 3/063

(57) ABSTRACT

A method for performing a pupillary stimulus threshold test on at least one eye of a test subject, the method comprising: delivering at least one visual stimulus to the at least one eye of the test subject, wherein the least one visual stimulus comprises a flash of light having an intensity and a duration; obtaining at least one pupillary response from the at least one eye of the test subject, wherein the at least one pupillary response corresponds to whether or not the test subject's pupil contracted in response to the at least one visual stimulus; and determining the lowest intensity of light that causes the test subject's pupil to contract based on the at least one pupillary response from the test subject.

### REFERENCE TO PENDING PRIOR PATENT APPLICATION

This patent application claims benefit of pending prior U.S. Provisional Patent Application Ser. No. 63/334,387, filed Apr. 25, 2022 by Diagnosys LLC and Jeffrey D. Farmer et al. for ENHANCED FULL-FIELD STIMULUS THRESHOLD (FST) AND PUPILLOMETRY SENSITIVITY THRESHOLD (PST) TESTS (Attorney's Docket No. DIAGNOSYS-18 PROV).

The above-identified patent application is hereby incorporated herein by reference.

### FIELD OF THE INVENTION

This invention relates to ophthalmic psychophysical diagnostic equipment in general, and more particularly to novel methods and apparatus for performing Full-field Stimulus Threshold (FST) and Pupillometry Sensitivity Threshold (PST) testing.

### BACKGROUND OF THE INVENTION

Ophthalmic psychophysical diagnostic equipment, such as that manufactured and sold by Diagnosys LLC of Lowell, MA, stimulates the eye (or eyes) of the test subject using flashes of light during the test and then the test subject responds by indicating whether or not they have seen (i.e., perceived) the flash of light. The response from the subject can be given verbally (e.g., “yes” or “no” or “no response”) and entered into the system by an observer (e.g., the clinician), or the response may be entered into the system by the test subject registering a response via a common input device (e.g., a keyboard or a subject response box, sometimes also referred to as a “button box”).

One such psychophysical test commonly performed on a test subject is the Full-field Stimulus Threshold (FST) test. Other common definitions of the FST acronym used by those skilled in the art include “Full-field Sensitivity Testing”, “Full-field Stimulus Testing” or other similar names to refer to the same type of ophthalmic test. Specifically, in the case of an FST test, the test is typically performed using a system which produces an audible tone that may (or may not) be followed by a flash of light (i.e., the “test flash” or “test flash stimulus”). Typically, a flash of light is produced contemporaneously with the audible tone, however, in a few instances during the course of the test, no flash of light is produced. This allows the clinician to check for a “false positive” response from the test subject (i.e., to control for the instance in which the test subject responds to the audible tone stimulus rather than the flash stimulus). After exposure to the audible tone stimulus (and, if applicable, the flash stimulus), the test subject then decides whether they saw (i.e., perceived) the flash of light, and the test subject inputs a corresponding “yes” or “no” into the system (e.g., using a button box or other input device). Then the test stimulus is repeated, i.e., another flash/tone combination is presented to the test subject. Although less commonly used, it is possible to perform an FST test that omits the audible sound (i.e., tone) stimulus that typically accompanies the flash stimulus. In such a modified FST test, the test subject is only required to provide a “yes” response when they actually perceive a test flash of light (and the test subject is not required to provide a “no” response when a flash of light is not perceived). Further ways to modify an FST test include providing the test subject with a single-button response box that simply represents a “yes” response when the button is pushed.

Typically, an FST test is conducted after the test subject has adapted to background light conditions (e.g., dark, or some constant background luminance of an arbitrary color) so that the flash threshold level at the prescribed level of light can quickly be measured. However, in other cases these tests can be started immediately after changing the background light (e.g., immediately after changing the background light from light to dark, or vice versa), thus measuring the process of visual sensitivity adaptation to the new background light conditions over a period of time. Such a test is typically referred to as a Dark Adaptometry (“DA”) test. The Dark Adaptometry test measures the same threshold as FST once the subject has fully adapted to the new background light conditions.

With FST tests such as those discussed above, the test flash stimulus is typically 1 millisecond or 200 milliseconds in length, however, if desired, the test flash stimulus can be shorter or longer. The system cycles through various flash luminance levels, in each case evoking a response from the subject with a goal of finding the flash level that is the lowest level of light the test subject can see, which is often referred to by those skilled in the art as the “threshold”. More particularly, the threshold is typically defined as the flash luminance level to which a subject responds “yes” 50% of the time and “no” 50% of the time, but other rules can be used to define the threshold, such as the minimum luminance level a subject could see 100% of the time during a test, 90% of the time, etc. Various ways to calculate the threshold value from the test subject responses have been used and are described in the literature, including approaches that vary the order in which flashes of different luminance levels are presented to the test subject. One of the most common techniques comprises presenting the flashes of light to the test subject in a bracketed, random organization, and fitting the total set of subject responses during a test to a Weibull, or other similar mathematical function, in order to calculate the threshold. The test may be conducted with the test subject's pupils dilated or undilated.

Other common attributes of a typical FST test include:
(1) a set maximum test time;
(2) automatically measured dark adaptation time prior to the test;
(3) the number, or target number, of reversals before finishing the test (a reversal is a test subject “yes” response followed by a test subject “no” response in sequence, or vice versa);
(4) display of the light luminance levels in dB, cd/m², and/or cd*s/m²;
(5) how much time the subject has to respond after presentation of the light/audible sound;
(6) the starting flash intensity used to begin the test;
(7) attributes of a fixation point (if used); and
(8) what audible sounds are used during the test, and other basic attributes for how the test is conducted and reported.

In prior art FST tests and DA tests employed today, the tests typically record between 20-50 subject responses. Typically, each response provided by a test subject is used in the calculation of the subject's threshold. The only exclusion criteria used to date to exclude a test subject's response from test results is an indication that the test subject's response differs “too much” from one or more of the other responses from the same test subject registered during the test. In many cases, such an exclusion is acceptable, e.g., if only one or two responses are clearly very inconsistent with, or different from, the majority of the other responses from that same test subject. However, in many cases the difference between a “potential outlier response” and the majority of the other responses from that test subject is not substantial, and hence, it is difficult (or impossible) to objectively determine if one or more of the responses can validly be excluded from the test results.

Aspects of how the test is conducted and reported help to judge the quality of the test taken by the test subject. During the test very high luminance flashes that the subject should see (based on their prior responses) are presented (i.e., to evaluate “false negative” responses), and in other cases no flash of light is presented at the time of an audible tone (evaluating ‘false positive’ responses). The overall quality of the FST test can further be assessed, either manually by a human (e.g., a clinician) or automatically using appropriate software, by various methods of analyzing the consistency of the test subject's responses across the whole test. These test quality attributes are typically included in the FST test report along with the threshold measured and other information from the test.

FIG. 1 is a schematic view showing an exemplary system 5 which may be used to perform FST tests on a test subject. System 5 generally comprises a stimulator 10 (e.g., a ganzfeld bowl stimulator, flash paddle or other similar device that produces light), a subject response box 15, a controller 20, a camera 25, and a computer 30 (with appropriate software). In use, stimulator 10 is configured to deliver a visual stimulus (e.g., a flash of light) to the eye(s) of a test subject, and the test subject registers whether the test subject perceived the visual stimulus using subject response box 15. Controller 20 is configured to control stimulator 10, and to convey test subject responses entered using controller 20 to computer 30. Appropriate software running on computer 30 is preferably used to analyze the responses and/or to modify the visual stimulus delivered to the test subject. Various physical setups are possible for system 5 and will be apparent to those skilled in the art in view of the present disclosure. However, the essential feature of prior art FST tests is that the light stimulator is positioned directly in front of the eyes of the subject and stimulates the visual field of the test subject's vision.

FIGS. 2 and 3 show exemplary FST test reports 35 generated using an FST system such as system 5 discussed above. More particularly, FIG. 2 is a test report obtained by performing an FST test on a test subject having normal vision, wherein one test result is shown on the graph. The X-axis of the graph is a range of test flash luminance levels, which is typically plotted in a Log cds/m² or dB scale, and the Y-axis is the probability of detection of the flash stimulus by the test subject, in %. Test subject responses are recorded by the system (e.g., after being input by the test subject using subject response box 15) and plotted based on the test subject responses to the flashes of light during a test. Probability values of 0% shown in FIGS. 2 and 3 represent flash luminance level(s) to which the test subject always responded “no” (i.e., the test subject indicated that they did not perceive the flash stimulus). Probability values of 100% shown in FIGS. 2 and 3 represent flash luminance level(s) to which the test subject always responded “yes”. Probability values other than 0% and 100% (i.e., probability values falling within the range >0% to <100%) result when the test subject is presented with stimuli having the same flash luminance level multiple times, and the test subject responds “yes” some of the time and “no” some of the time. Computer 30 (i.e., software running on computer 30) automatically calculates the threshold (in this case defined at the 50% probability of detection, but it could be defined as any % probability value), and records the threshold on the graph (FIGS. 2 and 3 show the threshold as indicated by a rectangle near the center of the graph at 40). Preferably, the software running on computer 30 also automatically presents the threshold value in a table.

FIG. 3 shows a test report 35 that is similar to the test report depicted in FIG. 2, however, FIG. 3 depicts a test report 35 resulting from a subject having low vision, and FIG. 3 also demonstrates that multiple test results may be plotted on the same graph.

FIGS. 4 and 5 show additional commercial examples of FST systems 45 that are generally similar to the exemplary system 5 discussed above, including the computer/software, controller, stimulator (with camera), and subject response box.

FIG. 6 shows three exemplary subject response boxes 15A, 15B and 15C. A two-button subject response box 15A is most commonly used, with one button of subject response box 15A being used to register a “yes” response, and a second button being used to register a “no” response, thereby creating a forced choice test. Response box 15B shows a more ergonomic version of two-button subject response box 15A. Alternatively, a single button response box 15C can be used instead of the two-button subject response boxes 15A and 15B, however, a single button response box 15C is not commonly utilized for FST testing.

FIG. 7 shows an exemplary Dark Adaptometry (“DA”) test report generated using a system such as system 5 discussed above. As previously discussed, the DA test measures intermediate and then final thresholds after the background light has been changed. The resulting curve shows the adaptation process of the visual system to the light change, and the final threshold in a DA test is a similar threshold measured in an FST test. In the case of the DA test report shown in FIG. 7, the test was done using both green and red flashes of light during the test, and those are plotted on the graph (green line data points are shown in FIG. 7 as triangles; red line data points are shown in FIG. 7 as ovals). The Y-axis of the graph of FIG. 7 represents a range of test flash luminance levels (which is typically plotted in a Log cds/m² or dB scale) and the X-axis represents time during the performing of the test. Test subject responses are recorded by the system (e.g., after being input by the test subject using subject response box 15) and plotted on the graph based on the test subject responses to the flashes of light during a test.

Prior art FST and DA tests currently being performed suffer from important limitations. Typically, the FST or DA test is conducted to determine the performance of a test subject's eye in perceiving very dim levels of light. The goal is not to measure the ability of the test subject's brain, personality, etc. to correctly respond with “yes” or “no” during the test. Rather, these tests rely on (and presume) a test subject's compliance with the testing parameters, the test subject's facility in interpretation of stimuli and general mental alertness during the test to watch for a flash of light, the test subject correctly interpreting whether they saw the light, and the test subject responding correctly. All of those criteria are not always met during an FST or DA test, and for some test subjects, are rarely met. In some cases, this lack of fidelity to the test parameters by the test subject makes a test completely unusable as a diagnostic measurement of the test subject's vision. While the goal is to measure the performance of the test subject's eye, the test unfortunately may end up being limited by the test subject's attention span, the test subject's mental or physical capabilities to comply during the test, etc. Measuring those non-vision-related conditions is not a goal of performing the test, and it can be difficult to ascertain their impact on the test.

Additional information relevant to the central question of “what is this test subject's eye's threshold to seeing the dimmest possible level of light?” can be collected while performing the tests, especially if there is no additional cost or physical requirement imposed on the test subject during the test. Such additional information can offer the potential of greatly improving the test's ability to answer that central question with greater accuracy, repeatability and statistical confidence.

Measuring the spontaneous variation of the pupil diameter and the pupillary light reflex in response to flashes of light (i.e., pupillometry) is an additional piece of information which can be relevant to answering the question of “what is this test subject's eye's threshold to seeing the dimmest possible level of light?”.

It is known that the eye's pupil changes its size based on the amount of light reaching the eye. The pupil of an eye in its natural state will generally constrict when the amount of light reaching the eye increases, and the pupil will generally dilate when the amount of light reaching the eye decreases. This well-known attribute has been used to create curves such as those shown in FIGS. 8 and 9, which are figures taken from Park et al., “Toward a Clinical Protocol for Assessing Rod, Cone, and Melanopsin Contributions to the Human Pupil Response”, Investigative Ophthalmology & Visual Science, Vol. 52, No. 9, 2011, pp. 6624-6635 (hereinafter “Park”). These figures show the pupil response to various flash intensities of light. The pupil constricts more in response to higher intensity flashes of light, and vice versa. As shown in the Park study, others have characterized a portion of the pupil constriction (i.e., pupil diameter in millimeters or % change of pupil diameter from just before the flash of light) response versus flash intensity as being log linear, as shown in FIG. 9. That pupillary response has been shown to be true for a wide array of eye types, both normal and diseased.

### SUMMARY OF THE INVENTION

The present invention comprises the provision and use of novel methods and apparatus for performing improved FST testing and measurement of a light stimulus threshold using pupillary responses to light.

In one preferred form of the present invention, there is provided a method for performing a pupillary stimulus threshold test on at least one eye of a test subject, the method comprising:
delivering at least one visual stimulus to the at least one eye of the test subject, wherein the least one visual stimulus comprises a flash of light having an intensity and a duration;
obtaining at least one pupillary response from the at least one eye of the test subject, wherein the at least one pupillary response corresponds to whether or not the test subject's pupil contracted in response to the at least one visual stimulus; and
determining the lowest intensity of light that causes the test subject's pupil to contract based on the at least one pupillary response from the test subject.

In another preferred form of the present invention, there is provided a method for performing a psychophysical stimulus threshold test on at least one eye of a test subject, the method comprising:
delivering at least one visual stimulus to the at least one eye of the test subject, wherein the at least one visual stimulus comprises a flash of light having an intensity and a duration;
obtaining at least one pupillary response from the at least one eye of the test subject, wherein the at least one pupillary response corresponds to whether or not the test subject's pupil contracted in response to the at least one flash of light;
obtaining at least one volitional response from the test subject, wherein the at least one volitional response corresponds to whether the test subject perceived a flash of light when the at least one flash of light was delivered to the at least one eye of the test subject; and
using the pupillary response to modify at least one testing condition of the psychophysical stimulus threshold test.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic view of an exemplary prior art system for performing an FST test on a test subject;

FIGS. 2 and 3 show exemplary test reports for FST tests performed on different test subjects;

FIGS. 4 and 5 are schematic views of exemplary systems for performing an FST test on a test subject;

FIG. 6 is a schematic view showing three exemplary subject response input devices;

FIG. 7 shows an exemplary Dark Adaptometry (“DA”) test report generated using a system such as the system of FIG. 1;

FIG. 8 shows Average Pupillary Light Reflexes (PLRs) from seven normal subjects to stimuli of different intensities in the dark with a) showing PLRs to red stimuli at 14 intensity levels; and b) showing PLRs to blue stimuli at 14 intensity levels;

FIG. 9 shows peak normalized pupil size versus stimulus intensity for seven normal subjects (small symbols) and their mean values (large symbols connected by thin lines);

FIG. 10a shows an exemplary screen shot of the camera's view of the pupil having a larger pupil before a flash stimulus;

FIG. 10b shows an exemplary screen shot of the camera's view of the pupil having a smaller pupil after a flash stimulus;

FIG. 11 shows an exemplary graph of pupil contraction versus flash intensity during an FST test;

FIG. 12 shows an exemplary graph of pupil contraction versus flash intensity during an FST test with flash intensities going to the maximum level of pupil contraction;

FIG. 13 shows an exemplary graph of pupil contraction versus flash intensity during an FST test, with select responses plotted on the graph;

FIG. 14 shows an exemplary graph illustrating a probability of detection method in accordance with the present invention;

FIG. 15 is a schematic view showing a novel system for performing a dichoptic FST test formed in accordance with the present invention; and

FIG. 16 is a schematic view showing a novel stimulator for performing a partial field FST test formed in accordance with the present invention.

### DETAILED DESCRIPTION OF THE PREFERRED EMBODIMENTS

The present invention comprises the provision and use of novel methods and apparatus for performing enhanced FST testing, including the use of information measured during the test regarding the reaction of the pupil to flashes of light during the FST test.

More particularly, the present invention generally comprises new methods and/or apparatus for determining a light threshold based on pupillary responses (e.g., contractions of the pupil) to flashes of light so as to measure a Pupillary Stimulus Threshold (“PST”). These methods also can be used in FST and DA testing, and can be used independently of one another, or in combination with one another. The new methods and apparatus of the present invention extend and improve the FST test (and hence, deliver a superior clinical result).

A first novel method according to the present invention comprises a technique for measuring a Pupillary Stimulus Threshold.

A second novel method according to the present invention utilizes information measured from the pupil reaction to flashes of light during the test to assist in judging the test subject's response in the FST test.

A third novel method according to the present invention combines the information from the Pupillary Stimulus Threshold and subject-response based FST threshold to create a combined threshold measurement.

### Pupillary Stimulus Threshold

Generally stated, a first novel method for performing a Pupillary Stimulus Threshold (PST) test on a test subject according to the present invention comprises (1) delivering at least one visual stimulus to the at least one eye of the test subject, wherein the least one visual stimulus comprises a flash of light having an intensity and a duration; (2) obtaining at least one pupillary response from the at least one eye of the test subject, wherein the at least one pupillary response corresponds to whether or not the test subject's pupil contracted in response to the at least one visual stimulus; and (3) determining the lowest intensity of light that causes the test subject's pupil to contract based on the at least one pupillary response from the test subject.

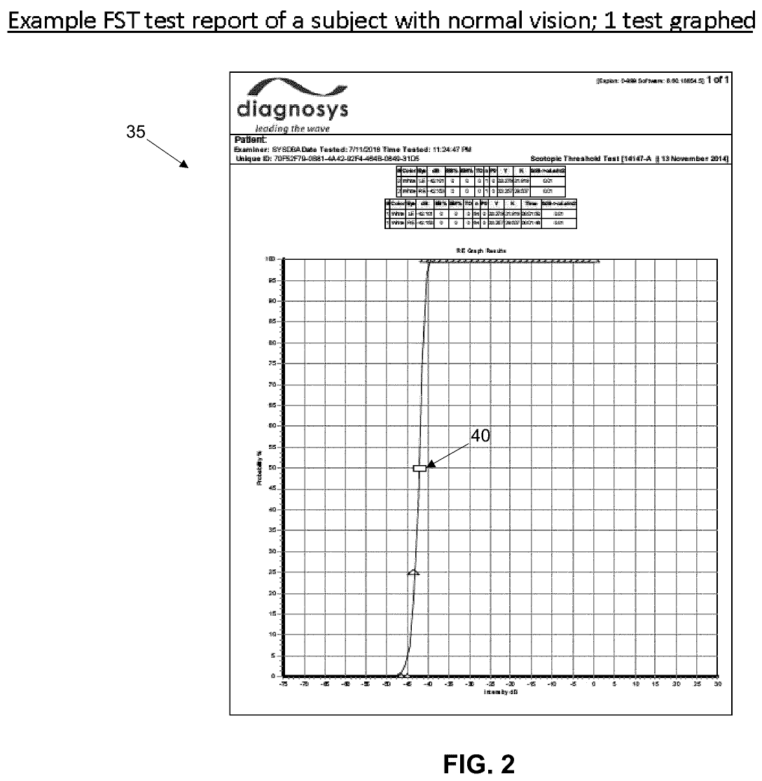

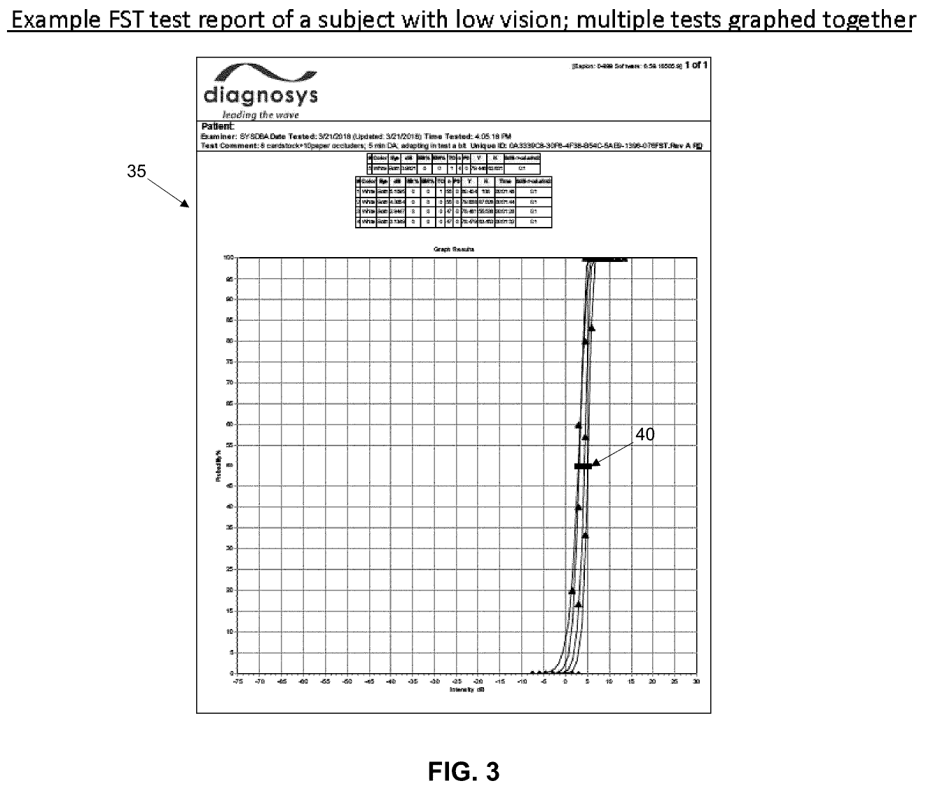

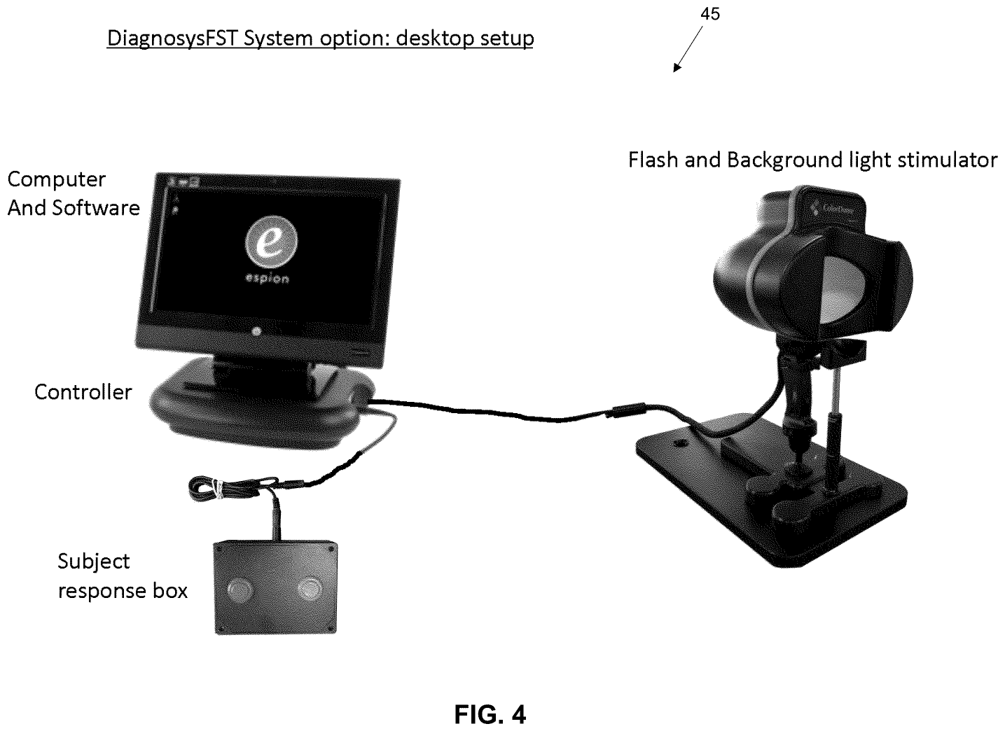

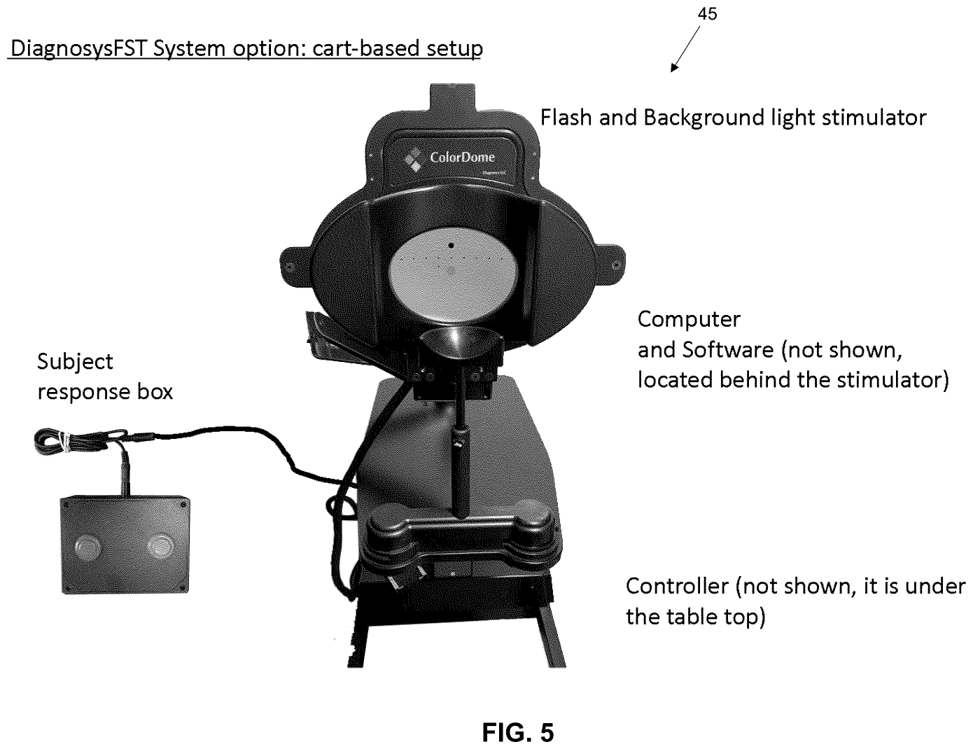

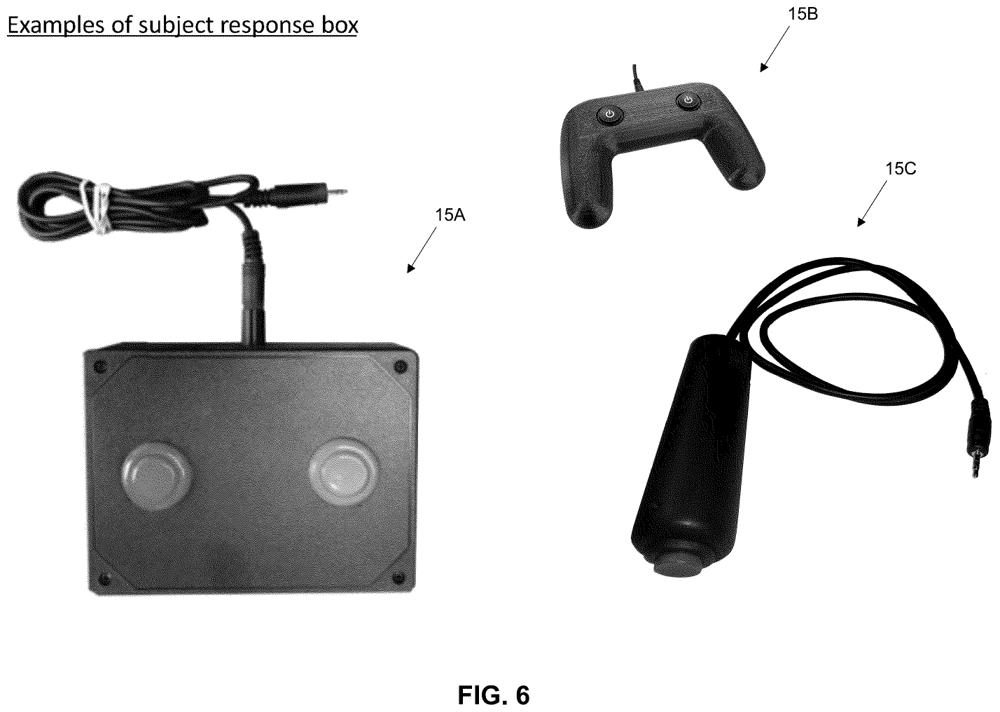

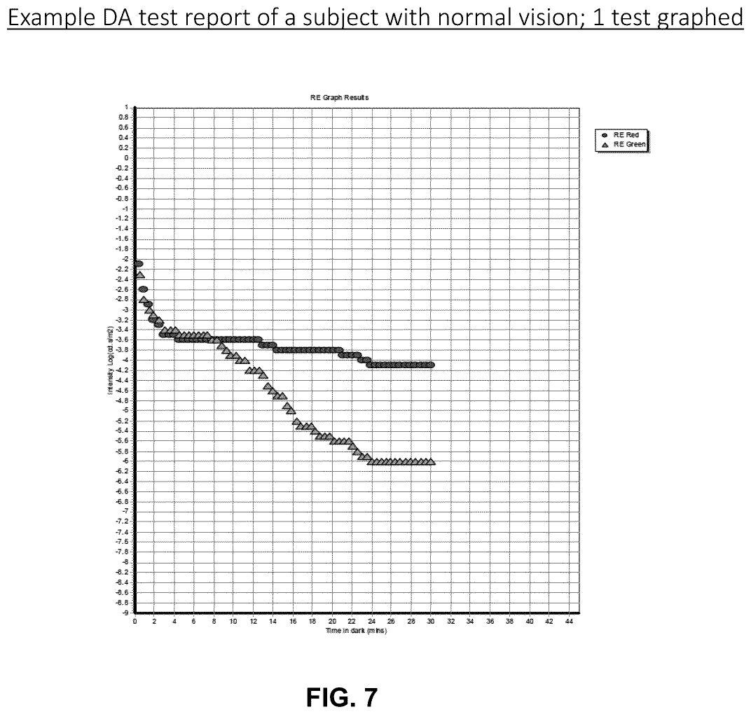

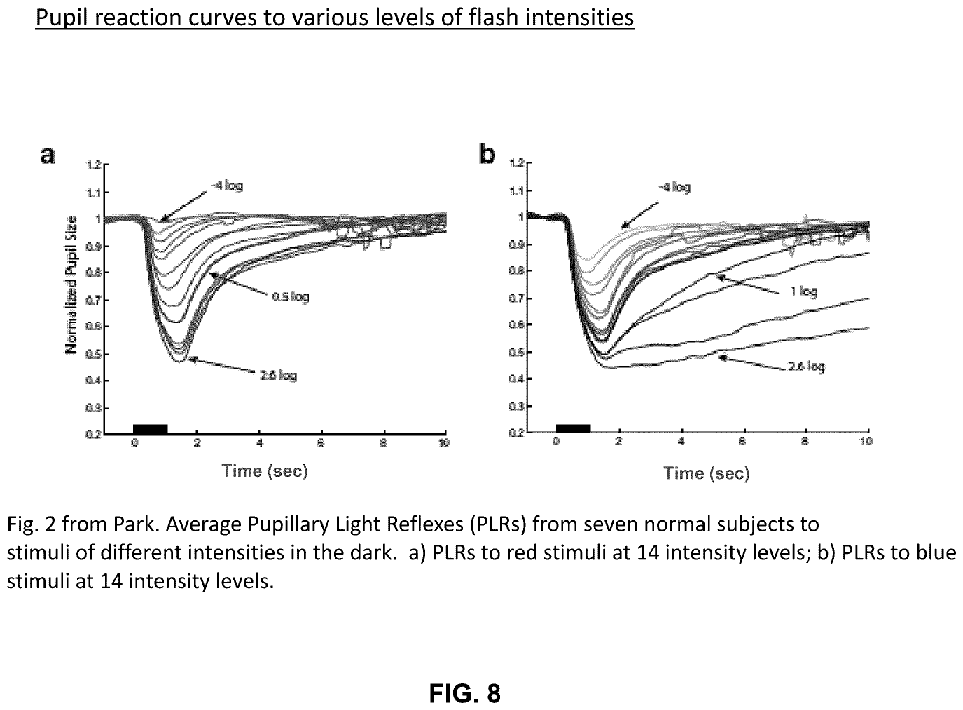

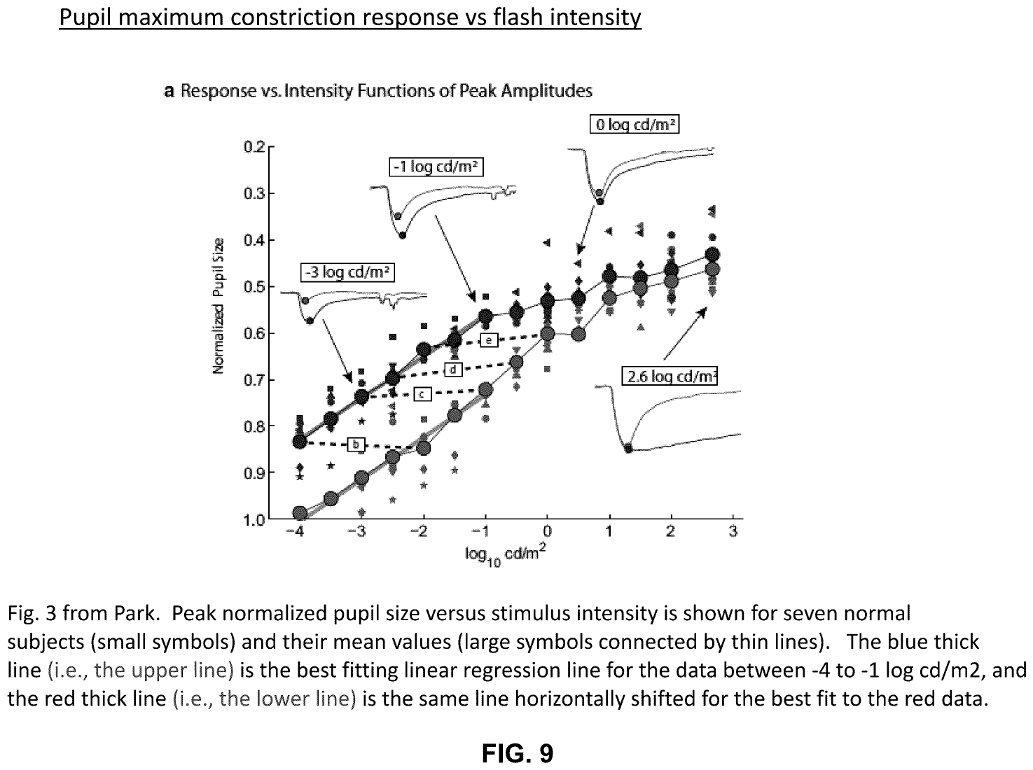

During a typical FST test, 20-50 paired audible tone/light flashes are presented to the test subject. FIG. 2 shows a typical graph of the test subject responses to the audible tone/light flash stimuli during the test. Generally, any specific flash strength may be, and oftentimes is, presented to a test subject multiple times over the duration of the test. Therefore, it is possible, (even typical) for test subject responses at some flash intensities to comprise a mix of “yes” and “no” responses, resulting in data points on the FST curve that are between 0% (i.e., all “no” responses) and 100% (i.e., all “yes” responses).

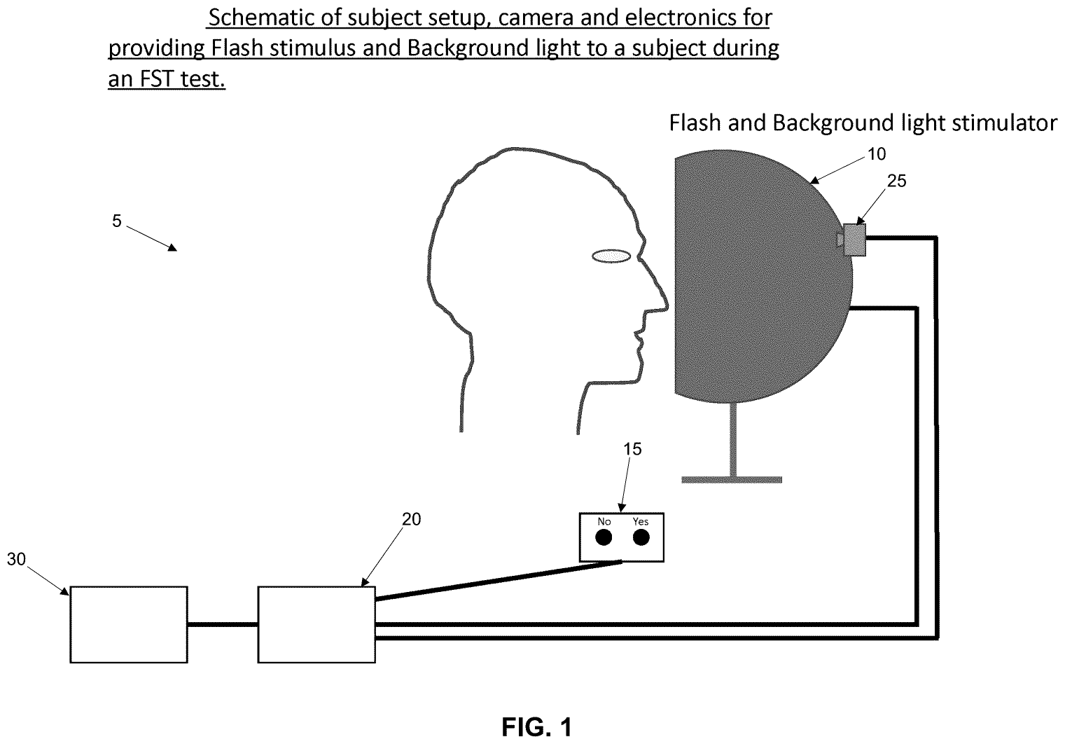

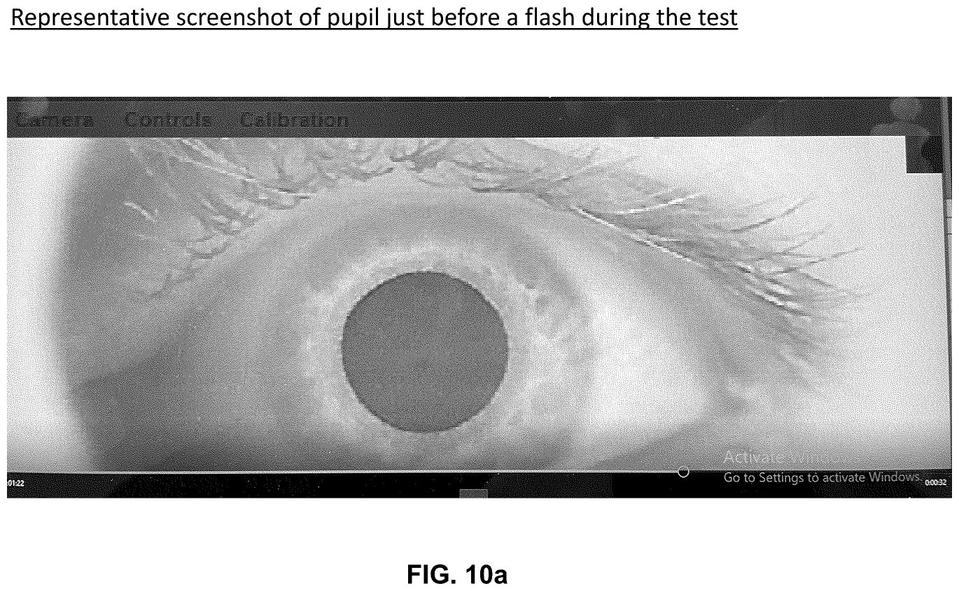

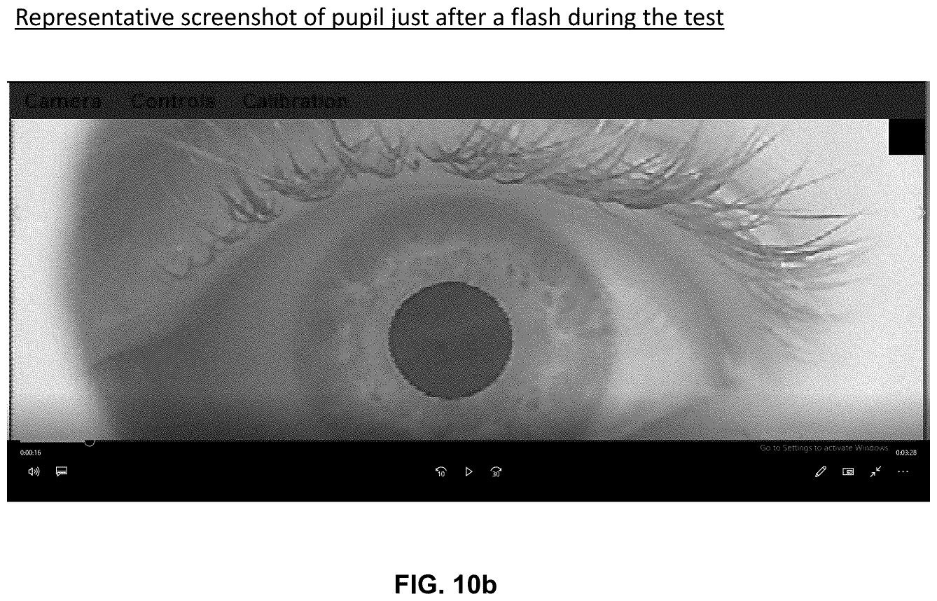

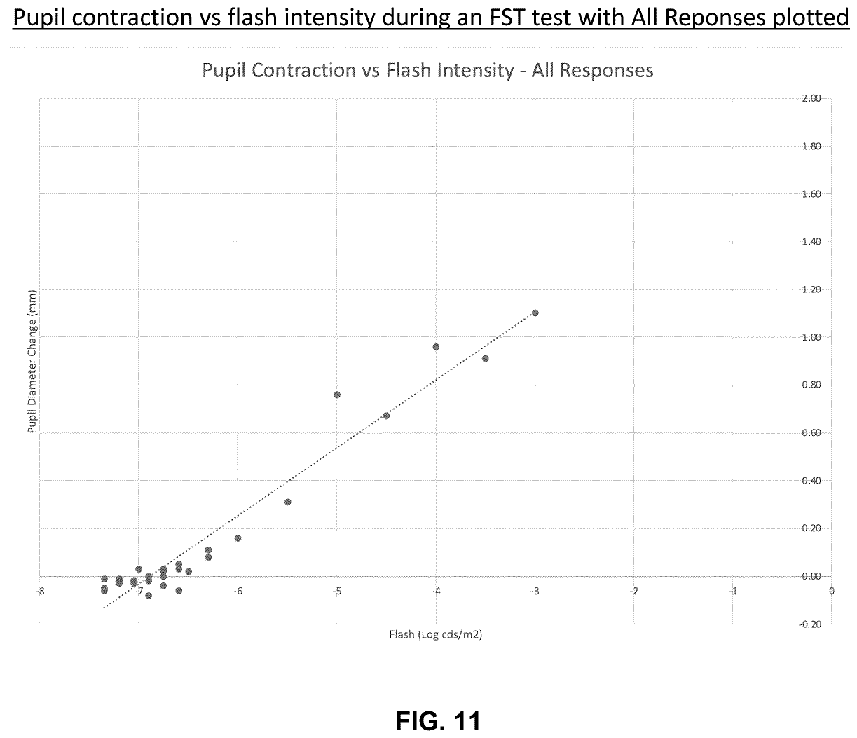

As shown in FIG. 1, where an FST system 5 comprises a camera for monitoring the test subject's eyes and a vision system (e.g., software running on computer 30 of system 5) for measuring the pupil's diameter (and other variables such as pupil shape, area, circumference, etc.) during the performance of the test, a pupil response curve can be generated in response to each flash of light presented during the FST test. Typical screen shots of the camera's view of the pupil are shown in FIG. 10a (showing a larger pupil before a flash stimulus) and FIG. 10b (showing a smaller pupil after a flash stimulus), with a dashed circular line representing the pupil size tracking mechanism of the pupillometer. If this pupil measurement is done for each flash of light during the test, and a maximum pupil constriction is measured following each flash of light, the resulting data may be plotted as shown in FIG. 11.

In FIG. 11, the Y-axis represents the maximum contraction of the pupil's diameter (i.e., the pupil diameter change) following a flash stimulus; the X-axis represents the flash intensity in Log cds/m². Flashes of light having a higher intensity are located to the right on the graph; larger pupil contractions are located toward the upper area of the graph. This is a typical response curve to one test. It should be appreciated that a significant portion of the response curve is linear, as has been reported in the past for similar tests. It should also be appreciated that the test presents a large number of flashes of light that are below the test subject's threshold (both psychophysically and pupillary), and these are the 10-15 points on the left side of the graph in FIG. 11. One point to note is that if this test were to have continued on to even lower intensity flashes of light, the purported pupil contractions would continue to be of the same magnitude as those around the −7 Log cds/m² flash level.

Thus it will be appreciated that an FST test can be performed on a test subject by observing changes in the size of the test subject's pupil in response to flash stimuli of varying intensities. More particularly, the method of performing an FST test according to this novel approach generally comprises the following steps:
(a) observing the size of the pupil of at least one eye of the test subject using a camera, whereby to obtain a measurement of the size of the unstimulated pupil;
(b) delivering at least one visual stimulus to the at least one eye of the test subject, wherein the at least one visual stimulus comprises a flash of light;
(c) observing the size of the pupil of the at least one eye of the test subject using the camera immediately after the visual stimulus is delivered to the at least one eye of the test subject, whereby to obtain a measurement of the size of the stimulated pupil;
(d) calculating the difference between the size of the unstimulated pupil and the size of the stimulated pupil so as to generate a pupil response measurement;
repeating steps (a) through (d) so as to generate a plurality of pupil response measurements, wherein each pupil response measurement is associated with a visual stimulus measurement, and further wherein the aggregated pupil response measurements and visual stimulus measurements comprise a data set; and
using the data set to calculate the full-field stimulus threshold for the at least one eye of the test subject.

It should also be appreciated that the pupil measuring system reports a negative pupil contraction in response to a flash stimulus. This means that the system is reporting the pupil of the test subject getting larger in response to the flash stimulus, and that is not possible (i.e., because the pupil will tend to constrict in response to a flash stimulus, rather than dilate). The 10-15 points in and around the −7 Log cds/m² flash level represent the “noise level” in the measurement capabilities of the pupillometry system, which is a very useful piece of information to have.

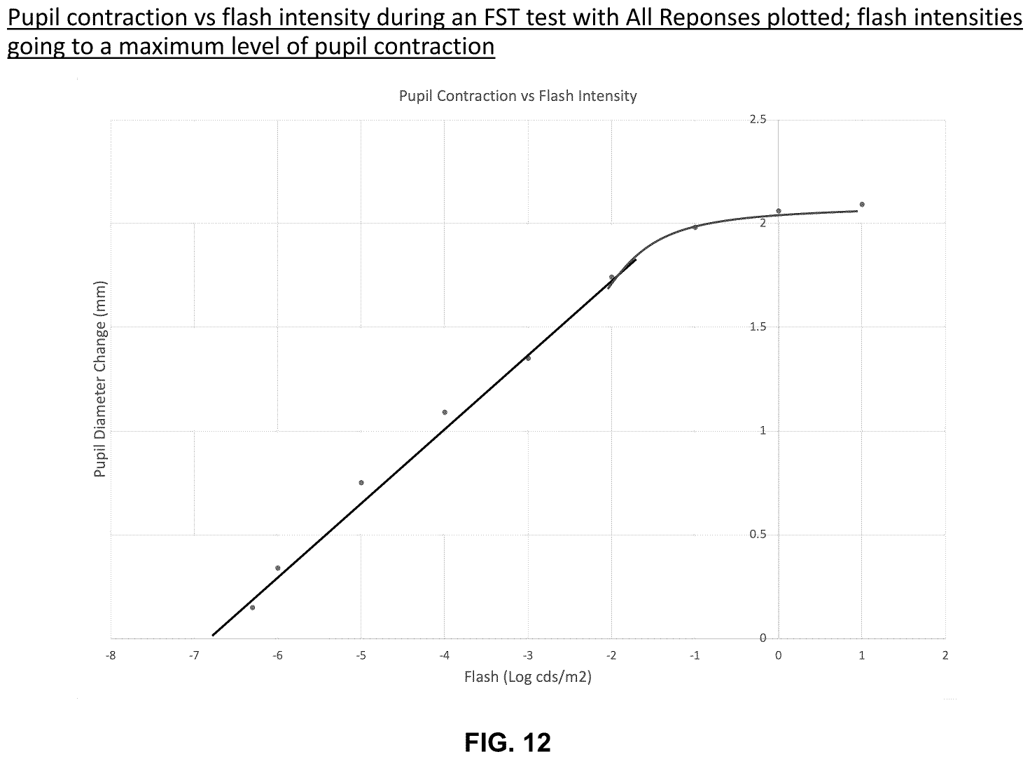

FIG. 12 shows a similar test performed on a subject, but a test in which the flash levels of the flash stimuli went much higher than the flash stimuli of the test results shown in FIG. 11. In the test depicted in FIG. 12, flash levels were as high as +1 Log cds/m² (compared to a maximum of −3 Log cds/m² in the prior test shown in FIG. 11). At bright enough flash intensities the pupil's maximum constriction will reach a point where it eventually becomes maximal, and hence, at higher flash intensities, little to no additional contraction will occur. This is a highly non-linear phase of the pupillary response to various flash intensities.

Based on these known and measured properties of the pupillary response to flashes of light during a test (which are general for a wide range of test subject's eyes), a novel method of calculating the Pupillary Stimulus Threshold may be realized in accordance with the present invention. The novel method utilizes information collected during the test regarding maximum pupillary contraction to each flash of light (i.e., the data in FIG. 11) and is performed as follows.

First, the noise level of the pupil contraction measurement is calculated based on data taken during the test. This calculation is based on two primary sources of data.

One source of data for use in calculating the noise level is the measurements of pupil diameter at a time just before each flash stimulus when the pupil diameter should be constant (but is not due to noise in the measurement method).

Generally stated, this method of obtaining data for use in calculating the noise level comprises (1) measuring the size of the pupil of the at least one eye of the test subject immediately before exposing the at least one eye of the test subject to a plurality of visual stimuli so as to generate a data set comprising a plurality of points reflecting the resting size of the pupil of the at least one eye of the patient immediately before exposing the at least one eye of the patient to the plurality of visual stimuli; (2) determining an average resting size of the pupil of the at least one eye of the patient immediately before a visual stimulus; and (3) determining the points that fall outside the average resting size of the pupil of the at least one eye of the patient, wherein the points that fall outside the average resting size of the pupil of the at least one eye of the patient are the noise level.

Another source of data for use in calculating the noise level is the data recorded for very dim flash stimuli, where contractions are measured to be “negative”. These two sources of data enable the noise level to be calculated, and hence, the “floor” of the test's ability to measure the smallest pupil contraction to a very dim flash of light. For the test shown in FIG. 11 the noise floor was calculated to be 0.06 mm.

Generally stated, this method of obtaining data for use in calculating the noise level comprises (1) measuring the size of the pupil of the at least one eye of the test subject immediately before exposing the at least one eye of the test subject to a plurality of visual stimuli so as to generate a data set comprising a plurality of points reflecting the resting size of the pupil of the at least one eye of the patient immediately before exposing the at least one eye of the patient to the plurality of visual stimuli; (2) exposing the at least one eye of the test subject to subject to a plurality of very dim visual stimuli; (3) measuring the size of the pupil of the at least one eye of the test subject immediately after exposing the at least one eye of the test subject to the plurality of very dim visual stimuli so as to generate a data set comprising a plurality of points reflecting the size of the pupil of the at least one eye of the patient immediately after exposing the at least one eye of the patient to the plurality of very dim visual stimuli; and (4) determining the point at which the pupil of the at least one eye of the test subject is larger after the visual stimulus is applied, which point corresponds to the smallest pupil contraction possible for the test subject, setting the noise level at which points below the noise level are considered noise.

Second, all pupil measurements below the noise floor level are removed from the data set, except for a single data point. The one data point that is retained is the pupil measurement that is below the noise and amongst all points below the noise floor, the data point was recorded at the brightest flash stimuli of those subset of measurements (i.e., the diameter of the pupil measured after delivery of the brightest flash of light amongst the data points below the noise floor level.

Third, the flash intensity of the one data point that was retained from the group below the noise floor is noted. That flash intensity can be referred to as F₀.

Fourth, all data points for maximum pupil contractions that were taken at flash intensities no greater than +3.5 Log cds/m² above F₀ are also kept in the final data set. Any data points taken at flashes of light exceeding +3.5 Log cds/m² above F₀ are removed from the final data set. This helps ensure that the final analysis is kept within the linear range of pupil responses, and avoids the non-linear region (shown in FIG. 12).

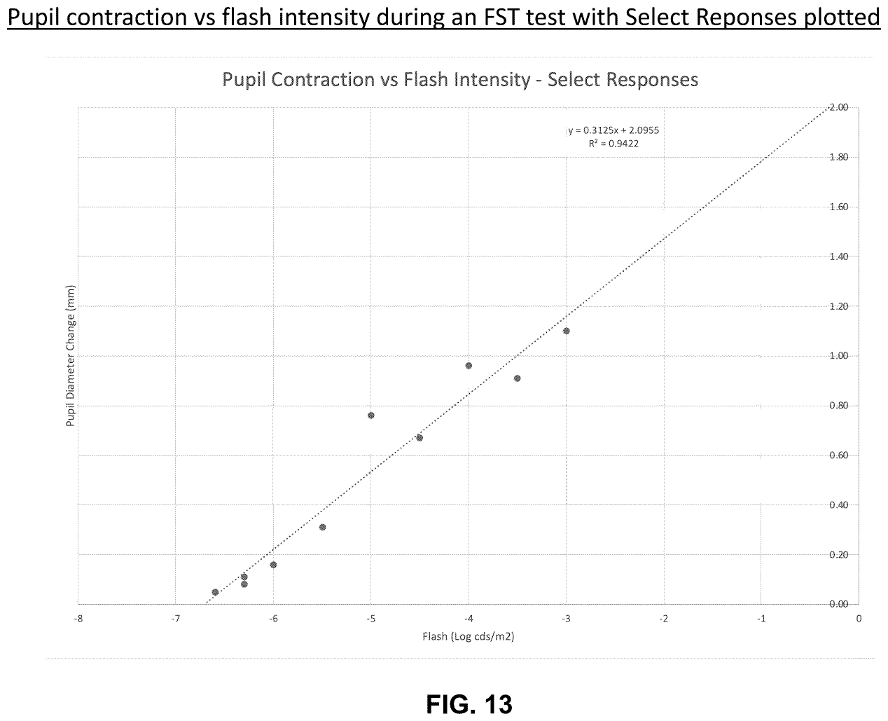

Fifth, after removing all data as described above, the remaining data is plotted on a Log scale and a linear curve is fit, as shown in FIG. 13. The flash intensity where the linear curve crosses the X-axis is defined as the Pupillary Sensitivity Threshold (“PST”). Stated another way, by determining the point at which the linear curve crosses the axis of the graph occupied by the intensity of the flashes of light, wherein the point at which the curve crosses the axis of the graph occupied by the intensity of the flashes of light comprises the lowest intensity of light that causes the test subject's pupil to contract, it is possible to define the Pupillary Sensitivity Threshold (PST).

In the example shown in FIGS. 11 and 13, the psychophysically measured FST of the subject was −6.96 Log cds/m². The PST was measured to be −6.70 Log cds/m². Both are important measures of function of the test subject's eye in measuring the lowest level of light that is detected by the test subject's eye.

It should also be appreciated that the method described above to measure the PST is general, and it is not required to be run at the same time as an FST. A series of light flashes similar to what is described above can be given during a test, with the PST calculated based on the method described above, with no FST test being performed. This is desirable in cases where it is only desirable to measure the pupil-based threshold. In this case, the flashes of light chosen can be optimized to only what is required to measure the PST using the novel method discussed above. By way of example but not limitation, in such a case, it is practical to simply conduct a test comprising a single series of flashes of light ranging from high to low (or vice versa) to provide the pupil reaction once per flash of light in a series that spans from below the threshold to above the linear portion of the pupil reaction curve. The PST-alone test then can be conducted even faster than when it is performed as part of an FST test (which test requires repeat flashes of light). A fully optimized PST-alone tests starts at a high intensity flash of light, and then progresses step-by-step to lower intensity flashes of light until the noise floor is reached, a repeat flash of light is done and then the test is ended after this fast sweep once through the flash stimulus levels.

A second novel method of calculating the PST can be performed using the same data from the test. Again, using the estimated noise floor of 0.06 mm pupil contraction (see FIG. 11), the second novel method categorizes all of the pupil responses into “yes” and “no” based on whether or not the measured pupil contraction is below the noise floor (i.e., a “no” response) or above the noise floor (i.e., a “yes” response). Given variability in the pupil measurement method, eye position and other variables, in practice, it is the case that, like the FST test, there are flash intensities with all “yes” responses, all “no” responses, or a mix of responses in the PST test. That is, each data point of the data set can be categorized into either a “yes” category or a “no” category, wherein the data points classified into the “yes” category are those data points corresponding to pupil contractions that are above the noise floor level, and wherein the data points classified into the “no” category are those data points corresponding to pupil contractions that are below the noise floor level.

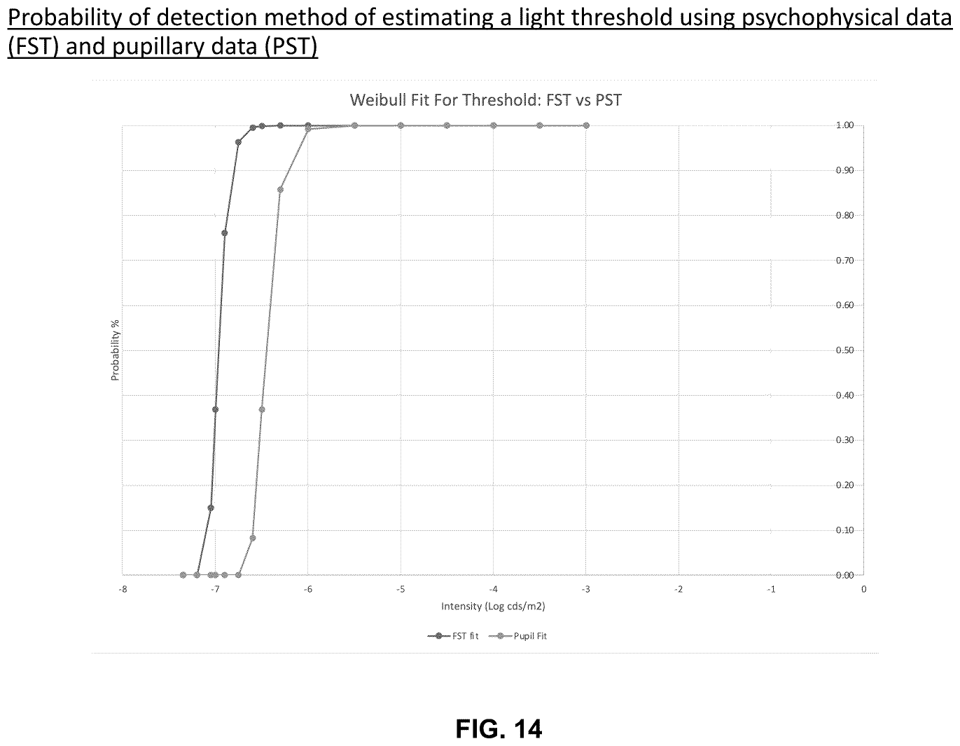

Given that, a second PST threshold representing the 50/50 chance of a measurable pupil reaction threshold can be calculated using a probability of detection plotting method and Weibull curve fit for the pupil data, as is shown in FIG. 14. Stated more generally, it is possible to determine an intensity threshold representing a N % chance of a measurable pupil contraction by determining where the Weibull fit crosses the axis representative of N % probability. In the case of the test results reported in FIG. 14, the FST threshold is measured to be −6.96 Log cds/m², as shown above. The PST using the probability of detection method for the same test was measured to be −6.50 Log cds/m². As can be seen in the graph of FIG. 14, the 0% probability of detection for the PST test is approximately −6.70 Log cds/m², matching the result of the first novel method of PST calculation discussed above, and representing the limit of detection for the eye to see light based on the PST calculation methods.

Generally stated, this method of obtaining data for use in calculating the PST comprises (1) performing a full-field stimulus threshold (FST) test on the at least one eye of the test subject so as to generate a data set comprising data points corresponding to measurements of the size of the pupil of the at least one eye of the test subject for visual stimuli of different intensities; (2) determining the maximum pupillary contraction of the at least one eye of the test subject based on the data set; (3) measuring the noise level of the pupillary contraction of the at least one eye of the test subject based on the data set; (4) categorizing each of the data points into either a “yes” category or a “no” category, wherein the data points classified into the “yes” category are those data points corresponding to visual stimuli that are above the noise level, and wherein the data points classified into the “no” category are those data points corresponding to visual stimuli that are below the noise level; (5) plotting the data points corresponding to the “yes” category on a graph such that the intensity of the visual stimulus occupies one axis and the probability associated with each visual stimulus occupies a second axis; (6) applying a Weibull curve fit to the data points plotted on the graph so as to derive a Weibull fit; and (7) determining an intensity threshold representing a 50% chance of a measurable pupil reaction threshold by determining where the Weibull fit crosses the axis representative of 0% probability.

Given that an FST system configured to measure PST comprises a camera and computer system (with appropriate software) monitoring the eye, the FST system can also be used to detect blinks by the test subject. Blinks that occur temporally near the time of the flash stimulus may disrupt the answer a test subject gives during the FST test and, therefore, that flash intensity can be repeated (or at least the data collected at that step in the test can be removed). Similarly for the PST test, blinks that occur temporally just after the flash stimulus can disrupt the measurement of the pupil response to the flash stimulus (which pupillary response occurs generally 0.5 to 1.5 seconds after the flash stimulus). In the PST test (or tests in which both FST and PST are being run at the same time) that flash intensity can be repeated (or at least the data collected at that step in the test can be removed from the PST calculations).

The FST test typically uses a pseudo-random algorithm for choosing a flash intensity for each stimulus presented to the test subject at any point in the test, and many of the flash intensities are presented to the test subject more than one time during the test. This is done for a number of reasons, including speed of the test being performed, and so as to avoid introducing a pattern to the flash intensities presented that the test subject (which might otherwise allow the test subject to memorize the stimulus pattern and therefore “cheat” on the test).

The improved FST methods described herein utilize the pupillary reaction to further improve the FST test. As discussed above, during the test both the test subject's volitional response (i.e., the response to the perception of the flash stimulus) and the test subject's physical pupillary response (i.e., the response of the test subject's pupil to the flash stimulus) are recorded and analyzed in real-time. Generally, a volitional “yes” response registered by the test subject should also generate a “yes” response for the physical pupillary measurement (as defined above), and similarly for the volitional “no” responses by the test subject, a “no” response for the physical pupillary measurement should be registered.

Where measured pupil contractions do and do not occur during the test, the improved system according to the present invention may take an action according to its decision algorithms based on this additional information, a feature which does not exist in prior art systems. By way of example but not limitation, such actions include: 1) repeat that flash intensity later in the test at least one additional time than otherwise would have occurred, 2) remove that data point from the FST and/or PST calculations, 3) factor the inconsistency in responses into a quality of test measurement factor (inconsistencies weighting the quality lower than would have otherwise been calculated without the information).

Stated more generally, the present invention comprises a novel method for performing a psychophysical stimulus threshold test on at least one eye of a test subject, the method comprising: (1) delivering at least one visual stimulus to the at least one eye of the test subject, wherein the at least one visual stimulus comprises a flash of light having an intensity and a duration; (2) obtaining at least one pupillary response from the at least one eye of the test subject, wherein the at least one pupillary response corresponds to whether or not the test subject's pupil contracted in response to the at least one flash of light; (3) obtaining at least one volitional response from the test subject, wherein the at least one volitional response corresponds to whether the test subject perceived a flash of light when the at least one flash of light was delivered to the at least one eye of the test subject; and (4) using the pupillary response to modify at least one testing condition of the psychophysical stimulus threshold test.

The foregoing novel method makes it possible, for the same flash of light delivered to the test subject, to analyze (i) the at least one pupillary response from the test subject, and (ii) the at least one volitional response from the test subject to determine consistencies and inconsistencies in the at least one pupillary response and the at least one volitional response, and to use the consistencies and inconsistencies to measure the quality of the psychophysical stimulus threshold test.

It should also be appreciated that all of these methods apply to a psychophysical test called Dark Adaptometry, which, as noted above, is a test that measures the threshold based on subject responses just like FST tests. The methods described herein of using pupil information to improve the tests applies to DA and FST while using what are called stair-case methods of flash intensity presentations, in addition to the pseudo-random method most commonly used today for FST.

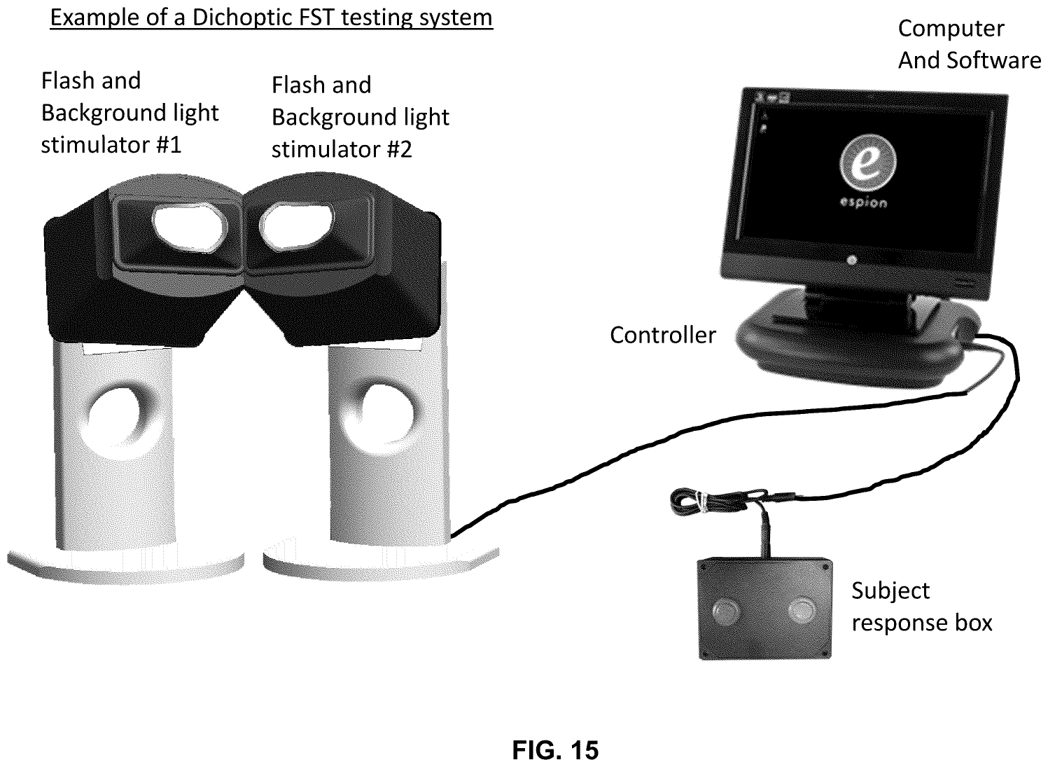

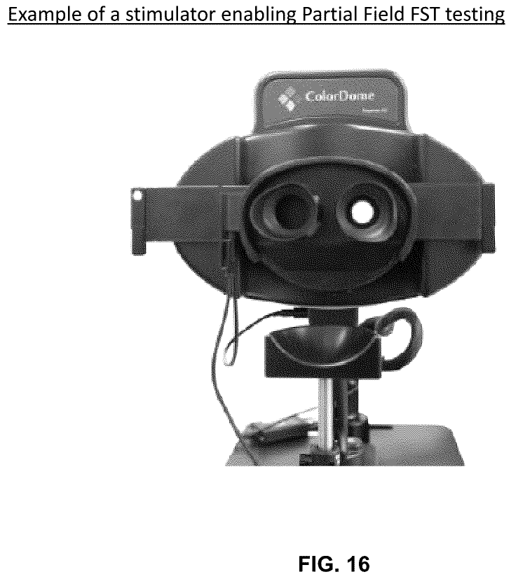

Finally, newer FST systems that utilize dichoptic stimulators (as shown in FIG. 15) and partial field FST systems (as shown in FIG. 16) utilize the pupillary methods exactly as described above for full-field, binocular stimulus systems.

### Modifications of the Preferred Embodiments

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. A method for performing a pupillary stimulus threshold test on at least one eye of a test subject, the method comprising:
delivering at least one visual stimulus to the at least one eye of the test subject, wherein the least one visual stimulus comprises a flash of light having an intensity and a duration;
obtaining at least one pupillary response from the at least one eye of the test subject, wherein the at least one pupillary response corresponds to whether or not the test subject's pupil contracted in response to the at least one visual stimulus; and
determining the lowest intensity of light that causes the test subject's pupil to contract based on the at least one pupillary response from the test subject.

2. The method of claim 1 wherein determining the lowest intensity of light that causes the test subject's pupil to contract comprises delivering a plurality of flashes of light having different intensities to the at least one eye of the test subject so as to generate a data set comprising data points corresponding to measurements of the diameter of the test subject's pupil after delivery of the plurality of flashes of light;
calculating a noise floor level of the pupillary responses of the at least one eye of the test subject, wherein the noise floor level comprises the lowest intensity of light that causes the test subject's pupil to contract;
removing all but one data point from the data points below the noise floor level, wherein the one data point retained from the data points below the noise floor level corresponds to the diameter of the pupil measured after delivery of the brightest flash of light amongst the data points below the noise floor level;
recording the intensity of the brightest flash of light;
removing data points corresponding to intensities of flashes of light above a predetermined intensity from the data set;
plotting the remaining data points on a graph such that the intensity of the flashes of light occupies one axis and the pupillary response associated with each flash of light occupies a second axis;
applying a linear curve fit to the data points plotted on the graph so as to generate a linear curve; and
determining the point at which the linear curve crosses the axis of the graph occupied by the intensity of the flashes of light, wherein the point at which the curve crosses the axis of the graph occupied by the intensity of the flashes of light comprises the lowest intensity of light that causes the test subject's pupil to contract.

3. The method of claim 2 wherein calculating the noise level comprises measuring the diameter of the pupil before a visual stimulus is delivered to the at least one eye of the test subject.

4. The method of claim 2 wherein calculating the noise level comprises measuring the diameter of the pupil in response to a dim visual stimulus delivered to the at least one eye of the test subject.

5. The method of claim 1 wherein determining the lowest intensity of light that causes the test subject's pupil to contract comprises delivering a plurality of flashes of light having different intensities to the at least one eye of the test subject so as to generate a data set comprising data points corresponding to measurements of the diameter of the test subject's pupil after delivery of the plurality of flashes of light;
calculating a noise floor level of the pupillary response of the at least one eye of the test subject based on the data set;
categorizing each data point of the data set into either a “yes” category or a “no” category, wherein the data points classified into the “yes” category are those data points corresponding to pupil contractions that are above the noise floor level, and wherein the data points classified into the “no” category are those data points corresponding to pupil contractions that are below the noise floor level;
plotting the data points corresponding to the “yes” category on a graph such that the intensity of the flashes of light occupies one axis and the probability of contraction associated with each flash of light occupies a second axis;
applying a Weibull curve fit to the data points plotted on the graph so as to derive a Weibull fit; and
determining an intensity threshold representing a N % chance of a measurable pupil contraction by determining where the Weibull fit crosses the axis representative of N % probability.

6. The method of claim 1 wherein a plurality of flashes of light are delivered to the at least one eye of the test subject, wherein the plurality of flashes of light comprise different intensities and different durations.

7. The method of claim 1 further comprising performing at least one of a full-field stimulus threshold (FST) test and a Dark Adaptometry (“DA”) test.

8. The method of claim 1 further comprising using a camera to detect a blink of the at least one eye of the test subject.

9. The method of claim 8 wherein upon detection of the blink at the same time as the flash, rejecting the pupillary response following the blink.

10. The method of claim 9 wherein after rejecting the at least one pupillary response, re-delivering the at least one visual stimulus to the at least one eye of the test subject.

11. The method of claim 1 wherein the intensity of the at least one flash of light varies each time a flash of light is delivered to the at least one eye of the test subject.

12. The method of claim 1 further comprising generating a graph, wherein the intensity of the at least one flash of light occupies one axis of the graph and the at least one pupillary response associated with the at least one flash of light occupies a second axis of the graph.

13. A method for performing a psychophysical stimulus threshold test on at least one eye of a test subject, the method comprising:
delivering at least one visual stimulus to the at least one eye of the test subject, wherein the at least one visual stimulus comprises a flash of light having an intensity and a duration;
obtaining at least one pupillary response from the at least one eye of the test subject, wherein the at least one pupillary response corresponds to whether or not the test subject's pupil contracted in response to the at least one flash of light;
obtaining at least one volitional response from the test subject, wherein the at least one volitional response corresponds to whether the test subject perceived a flash of light when the at least one flash of light was delivered to the at least one eye of the test subject; and
using the pupillary response to modify at least one testing condition of the psychophysical stimulus threshold test.

14. The method of claim 13 wherein at least one of (i) the at least one pupillary response from the test subject, and (ii) the at least one volitional response from the test subject is used to determine the lowest intensity of light that causes the test subject's pupil to contract in response to the at least one flash of light.

15. The method of claim 13 wherein modifying at least one testing condition comprises delivering an additional flash of light to the at least one eye of the test subject, wherein the additional flash of light is a repeat of a previously delivered flash of light to the at least one eye of the test subject.

16. The method of claim 13 further comprising discarding at least one of (i) the at least one pupillary response from the test subject, and (ii) the at least one volitional response from the test subject prior to determining the lowest intensity of light that causes the test subject's pupil to contract in response to the at least one flash of light.

17. The method of claim 13 further comprising for the same flash of light delivered to the test subject, analyzing (i) the at least one pupillary response from the test subject, and (ii) the at least one volitional response from the test subject to determine consistencies and inconsistencies in the at least one pupillary response and the at least one volitional response, wherein the consistencies and inconsistencies are used to measure the quality of the psychophysical stimulus threshold test.

18. The method of claim 13 further comprising using a camera to detect a blink of the at least one eye of the test subject.

19. The method of claim 18 wherein upon detection of the blink, rejecting the at least one pupillary response following the blink.

20. The method of claim 19 wherein after rejecting the at least one pupillary response, re-delivering the at least one flash of light to the at least one eye of the test subject.
